(19) United States
(12) Patent Application Publication
(10) Pub. No.: US 2025/0064327 A1
(43) Pub. Date: Feb. 27, 2025

(54) BIOSENSOR SYSTEM AND METHOD FOR NON-INVASIVE MEASUREMENT AND PREDICTION OF BLOOD GLUCOSE AND BLOOD PRESSURE

(71) Applicant: MAVOIX TECHNOLOGY SOLUTIONS PRIVATE LIMITED, Bangalore (IN)

(72) Inventors: Suprokash Goswami, Bangalore (IN); Rajat Agrawal, Kanpur (IN)

(21) Appl. No.: 18/810,609

(22) Filed: Aug. 21, 2024

(30) Foreign Application Priority Data
Aug. 21, 2023 (IN) 202341056053

(51) Int. Cl. A61B 5/0205; A61B 5/00; A61B 5/145; A61B 5/1455

(52) U.S. Cl. CPC A61B 5/0205; A61B 5/0022; A61B 5/14532; A61B 5/1455; A61B 5/7203; A61B 5/726; A61B 5/7264; A61B 5/742; A61B 5/7475

(57) ABSTRACT

The present invention discloses a biosensor system designed for non-invasive measurement and prediction of blood glucose and blood pressure levels. The system comprises a biosensor module, a processing module, a convolutional neural network (CNN) module, a display module, a user interface module, and a control module. The biosensor module incorporates optical spectroscopic techniques to capture physiological signals, which are preprocessed to eliminate noise and optimize for subsequent analysis. A scalogram image is generated from the preprocessed signals, and the processing module further processes the image. The CNN module utilizes the processed scalogram image to accurately measure and predict blood glucose and blood pressure levels. The system offers a user-friendly interface displayed on a screen, enabling users to interact and view the predicted results. The proposed method is non-invasive, relying on capturing and analyzing the physiological signals to provide reliable and convenient monitoring of blood glucose and blood pressure levels.

### CROSS REFERENCE TO RELATED APPLICATION(S)

This application claims the priority to and incorporates by reference the entire disclosure of Indian patent application bearing No. 202341056053 filed on Aug. 21, 2023 and titled “A BIOSENSOR SYSTEM AND METHOD FOR NON-INVASIVE MEASUREMENT AND PREDICTION OF BLOOD GLUCOSE AND BLOOD PRESSURE”.

### FIELD OF INVENTION

The present invention relates to the field of healthcare technology and specifically to a non-invasive device and method for measuring and predicting blood glucose and blood pressure levels using physiological signals analysis and Convolutional Neural Network (CNN) modeling.

### BACKGROUND OF THE INVENTION

Traditional methods for measuring blood glucose and blood pressure have relied on invasive techniques that may cause discomfort and inconvenience to the users. Blood glucose measurement often involves finger pricking to obtain a blood sample, which can be painful and discourages regular monitoring. Similarly, blood pressure measurement typically requires the use of cuff-based devices, which may restrict mobility and require proper positioning and calibration for accurate readings. These conventional methods present limitations in terms of user comfort, convenience, and real-time monitoring capabilities.

In recent years, several non-invasive approaches have been explored to overcome these limitations and provide more user-friendly alternatives for measuring blood glucose and blood pressure. Optical spectroscopy techniques have been employed to capture physiological signals and analyze blood components using light absorption or scattering properties. However, these methods often require complex calibration processes and may be influenced by factors such as skin pigmentation and ambient light conditions, leading to potential inaccuracies in the measurements.

Another non-invasive approach involves impedance-based measurements, which rely on the electrical properties of body tissues to estimate blood glucose and blood pressure levels. These methods utilize sensors or electrodes to measure electrical impedance changes in the body, but they may suffer from limited accuracy due to variations in tissue properties and the influence of external factors such as body composition and hydration levels.

Pulse wave analysis is another technique used for non-invasive blood pressure measurement. It involves analyzing the characteristics of the arterial pulse waveform to estimate blood pressure parameters. While this method provides continuous monitoring capabilities, it relies on accurate waveform analysis and calibration against cuff-based measurements, which may introduce potential errors and require periodic recalibration.

Furthermore, advancements in machine learning and artificial intelligence have enabled the development of predictive models for blood glucose and blood pressure estimation. Convolutional Neural Networks (CNNs) have been successfully applied in various healthcare applications, including medical image analysis and physiological signal processing. These deep learning techniques extracts features from input signals and make accurate predictions based on the learned patterns and relationships within the data.

Despite these advancements, there remains a need for an integrated and user-friendly non-invasive device and method that can accurately measure and predict blood glucose and blood pressure levels in real-time. Such a device should overcome the limitations of existing techniques by providing a comfortable and convenient monitoring experience, while ensuring high accuracy and reliability in the measurements. The present invention aims to fulfill this need by combining physiological signals analysis with CNN modeling, enabling simultaneous and non-invasive monitoring of blood glucose and blood pressure levels.

### SUMMARY OF THE INVENTION

In light of the disadvantages mentioned in the previous section, the following summary is provided to facilitate an understanding of some of the innovative features unique to the present invention and is not intended to be a full description. A full appreciation of the various aspects of the invention can be gained by taking the entire specification and drawings as a whole.

The present invention relates to a biosensor system, a method, and an apparatus for non-invasive measurement and prediction of blood glucose and blood pressure levels. The invention comprises various components designed to capture, process, analyse and transmit physiological signals.

A biosensor system for non-invasive measurement and prediction of blood glucose and blood pressure levels is disclosed. The biosensor system comprises of a biosensor module configured to capture physiological signals and preprocess the captured signals to eliminate noise and optimize for subsequent analysis. A processing module is configured to extract relevant information from the preprocessed physiological signals using a processing technique and a convolutional neural network (CNN) module is configured to measure and predict blood glucose and blood pressure levels based on the extracted information. A display module comprising a screen and a user interface module configured to receive one or more user commands and display information on the screen. Further, a control module is configured to control the operation of the biosensor apparatus.

A method for non-invasive measurement and prediction of blood glucose and blood pressure levels using a physiological signals captured by a biosensor apparatus is disclosed. The method comprises the steps of placing a finger on the biosensor apparatus to capture the physiological signals, continuously capturing the physiological signals for a predetermined duration, initiating an API call to securely store the captured signal in a cloud-based database, retrieving the captured signal from the cloud-based database, pre-processing the captured signal to eliminate noise and optimize for subsequent analysis, extracting relevant information from the pre-processed signal, employing a Convolutional Neural Network (CNN) module to measure and predict blood glucose and blood pressure levels using the extracted information and displaying the measured and predicted blood glucose and blood pressure levels on a screen of the biosensor apparatus.

An apparatus for non-invasive measurement and prediction of blood glucose and blood pressure levels is disclosed. The apparatus comprises of a biosensor module incorporating optical spectroscopic techniques for capturing physiological signals, a microcontroller module with networking capabilities for signal processing, analysis and transmission, a display screen module for presenting measurement results, a LiPo battery module for power supply and an enclosure module for housing and protecting the components.

According to an embodiment of the present invention, the biosensor module is configured to transmit the captured physiological signals, pre-processed data, and prediction results to a cloud-based server for remote patient monitoring. This allows healthcare providers to remotely access and monitor patients' health data as healthcare IT service.

Overall, the present invention offers a non-invasive and user-friendly approach to monitor blood glucose and blood pressure levels, providing accurate measurements and predictions for improved healthcare management.

This summary is provided merely for purposes of summarizing some example embodiments, so as to provide a basic understanding of some aspects of the subject matter described herein. Accordingly, it will be appreciated that the above-described features are merely examples and should not be construed to narrow the scope or spirit of the subject matter described herein in any way.

### BRIEF DESCRIPTION OF THE DRAWING

FIG. 1 illustrates a block diagram of an embodiment of a biosensor system for non-invasive measurement and prediction of blood glucose and blood pressure levels according to an embodiment of the present disclosure.

FIG. 1a illustrates a block diagram of an embodiment of a Convolutional Neural Network (CNN) module for non-invasive measurement and prediction of blood glucose and blood pressure levels according to an embodiment of the present disclosure.

FIG. 2 illustrates a block diagram of an embodiment of an apparatus for non-invasive measurement and prediction of blood glucose and blood pressure levels according to an embodiment of the present disclosure.

FIG. 3 illustrates a flowchart illustrating a method steps for non-invasive prediction of blood glucose and blood pressure levels according to an embodiment of the present disclosure.

FIG. 4 illustrates a schematic diagram illustrating an emitter unit and a detector unit of a biosensor module according to an embodiment of the present disclosure.

FIGS. 5a to 5c illustrates a schematic diagram illustrating an enclosure of the apparatus for non-invasive measurement and prediction of blood glucose and blood pressure levels according to an embodiment of the present disclosure.

FIG. 6 illustrates a schematic diagram illustrating an overall workflow of the biosensor system for non-invasive measurement and prediction of blood glucose and blood pressure levels according to an embodiment of the present disclosure.

FIGS. 7a and 7b illustrates a diagram illustrating a first convolutional layer operation of Convolutional Neural Network (CNN) module according to an embodiment of the present disclosure.

FIGS. 8a and 8b illustrates a graphical representation of output of an activation layer of Convolutional Neural Network (CNN) module according to an embodiment of the present disclosure.

FIG. 9 illustrates a schematic diagram illustrating a max pooling operation of Convolutional Neural Network (CNN) module according to an embodiment of the present disclosure.

FIG. 10 illustrates a schematic diagram illustrating blocks of a convolutional layer, a ReLU activation layer and a MaxPooling layer according to an embodiment of the present disclosure.

FIG. 11 illustrates a schematic diagram illustrating pooling layers, flatten, fully connected layers and output layer according to an embodiment of the present disclosure.

FIG. 12 illustrates a schematic diagram illustrating the function of dropout layer according to an embodiment of the present disclosure.

FIG. 13 is a table representing all the layers, their return shapes and the number of training parameters according to an embodiment of the present disclosure.

FIGS. 14a and 14b illustrates a protype of the apparatus for non-invasive measurement and prediction of blood glucose and blood pressure levels according to an embodiment of the present disclosure.

FIGS. 15a to 15c is a graphical representation of comparison of levels of blood glucose and blood pressure between the existing products in the market and the apparatus of the present invention.

### DETAILED DESCRIPTION OF THE DRAWINGS

For the purpose of promoting an understanding of the principles of the disclosure, reference will now be made to the embodiment illustrated in the figures and specific language will be used to describe them. It will nevertheless be understood that no limitation of the scope of the disclosure is thereby intended. Such alterations and further modifications in the illustrated system, and such further applications of the principles of the disclosure as would normally occur to those skilled in the art are to be construed as being within the scope of the present disclosure.

The terms “comprises”, “comprising”, or any other variations thereof, are intended to cover a non-exclusive in moduleclusion, such that a process or method that comprises a list of steps does not include only those steps but may include other steps not expressly listed or inherent to such a process or method. Similarly, one or more devices or sub-systems or elements or structures or components preceded by “comprises . . . a” does not, without more constraints, preclude the existence of other devices, sub-systems, elements, structures, components, additional devices, additional sub-systems, additional elements, additional structures, or additional components. Appearances of the phrase “in an embodiment”, “in another embodiment” and similar language throughout this specification may, but not necessarily do, all refer to the same embodiment.

Unless otherwise defined, all technical and scientific terms used herein have the same meaning as commonly understood by those skilled in the art to which this disclosure belongs. The system, methods, and examples provided herein are only illustrative and not intended to be limiting.

A biosensor system 100 for non-invasive measurement and prediction of blood glucose and blood pressure levels is disclosed.

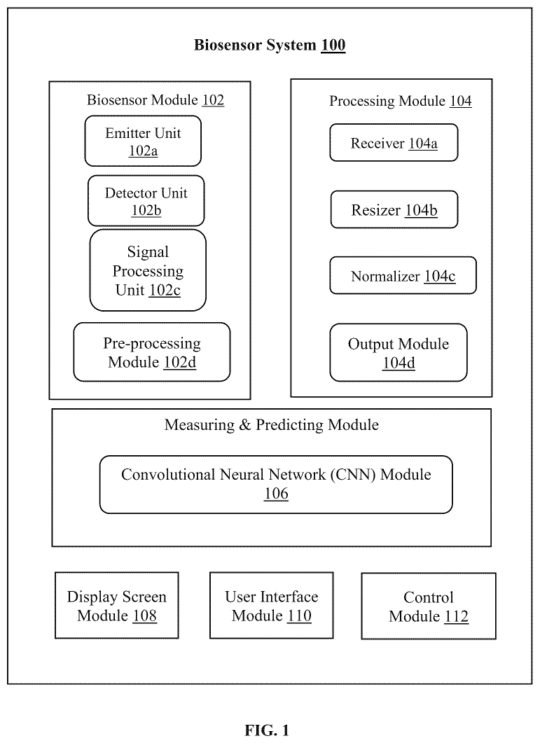

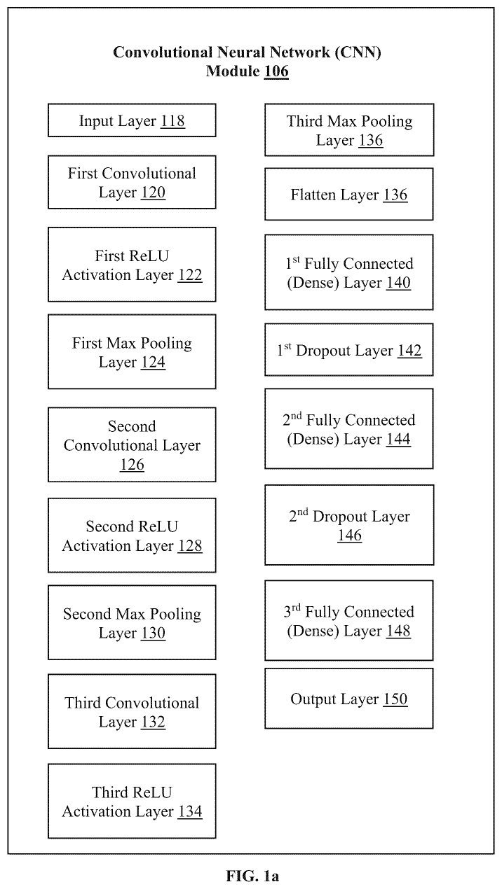

FIG. 1 illustrates a block diagram of an embodiment of a biosensor system 100 for non-invasive measurement and prediction of blood glucose and blood pressure levels according to an embodiment of the present disclosure. The biosensor system 100 comprises of a biosensor module 102 configured to capture signals comprising physiological signals and preprocess the captured signals to eliminate noise and optimize for subsequent analysis. A processing module 104 is configured to extract relevant information from the preprocessed PPG signal using a processing technique and a convolutional neural network (CNN) module 106 is configured to measure and predict blood glucose and blood pressure levels based on the extracted information. A display module comprising a screen and a user interface module 110 configured to receive one or more user commands and display information on the screen. Further, a control module 112 is configured to control the operation of the biosensor apparatus.

According to an embodiment of the present invention, the biosensor module 102 incorporates optical spectroscopic techniques for capturing physiological signals. The biosensor module 102 comprises of an emitter unit 102a configured to emit light wavelengths in the range of 660 nm to 1100 nm onto a target region of a user's body and a detector unit 102b positioned adjacent to the emitter unit 102a and configured to receive the light after it has reflected through the target region. It further comprises of a signal processing unit 102c operatively coupled to the detector unit 102b, configured to process the received light signals and generate physiological signals. A pre-processing module 102d is configured to eliminate noise and optimize the physiological signals for subsequent analysis. Further, the emitter unit 102a comprises of a plurality light sources and the detector unit comprises one or more photodetectors sensitive to the emitted light wavelengths. Also, the signal processing unit 102c further comprises a spectral analysis module for analysing the received light signals to extract physiological information.

According to an embodiment of the present invention, the biosensor module 102 preprocesses the signals to generate scaled signals for generating a scalogram image. The preprocessing steps comprise of scaling the signals from an original range to a new range of 0 to 255. Next, implementing a continuous wavelet transform on the scaled signals using a Complex Gaussian Derivative Wavelet with 8th-order derivatives. Further, obtaining coefficients and frequencies from the continuous wavelet transform and generating a scalogram image by mapping the obtained coefficients onto a pixel grid. Finally, save the scalogram image.

According to an embodiment of the present invention, the processing technique implemented by the processing module 104 is configured to process the scalogram image. The processing module 104 comprises of a receiver 104a configured to receive the scalogram image and a resizer 104b is configured to resize the scalogram image array to a new shape of (255, 255, 3). It further comprises a normalizer 104c configured to normalize the scalogram image array. An output module 104d configured to output the processed scalogram image array.

FIG. 1a illustrates a block diagram of an embodiment of a Convolutional Neural Network (CNN) module 106 for non-invasive measurement and prediction of blood glucose and blood pressure levels according to an embodiment of the present disclosure. According to an embodiment of the present invention, the Convolutional Neural Network (CNN) module 106 is configured to measure and predict blood glucose and blood pressure levels. The CNN module 106 comprises of an input layer 118 configured to receive a preprocessed scalogram image array having an input shape of (255, 255, 3), representing width, height, and number of color channels. A convolutional layer configured to apply a set of 16 filters of size 3×3 to the input image array, with a stride of 1 and valid padding, producing an output of size (253, 253, 16). An activation layer is configured to apply a rectified linear unit (ReLU) activation function to the output of the convolutional layer, producing an output of size (253, 253, 16). A max pooling layer is configured to perform max pooling with a window size of 2×2, a stride of 2, and valid padding on the output of the activation layer, producing an output of size (126, 126, 16). Further it comprises of two blocks constituting three layers each, Convolution, Activation, Max Pooling layer respectively with their respective parameters. The output of the trailing block is flattened and connected to the first fully connected layer 140 with 1024 neurons and ReLU activation function followed by a dropout layer with dropout rate of 20%. A second fully connected layer 144 with 512 neurons and ReLU activation function connected with the second dropout layer 146 with a dropout rate of 20%. Further a third fully connected layer 148 with 128 neurons and ReLU activation function connected with the final output layer 150 with 3 neurons and a linear activation function representing blood glucose, systolic blood pressure, and diastolic blood pressure predictions.

A method for non-invasive measurement and prediction of blood glucose and blood pressure levels using a physiological signals captured by a biosensor apparatus is disclosed.

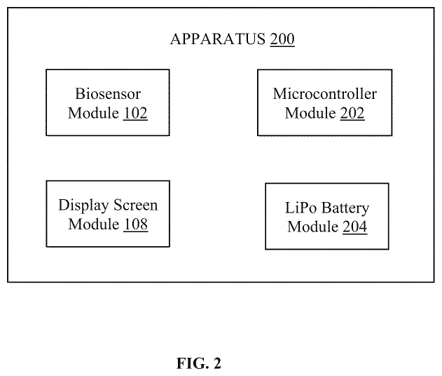

FIG. 3 illustrates a flowchart illustrating a method steps for non-invasive measurement and prediction of blood glucose and blood pressure levels according to an embodiment of the present disclosure. The method comprises the steps of placing a finger on the biosensor apparatus to capture the physiological signals at step 302, continuously capturing the physiological signals for a predetermined duration at step 304, initiating an API call to securely store the captured signal in a cloud-based database at step 306, retrieving the captured signal from the cloud-based database at step 308, pre-processing the captured signal to eliminate noise and optimize for subsequent analysis at step 310, extracting relevant information from the pre-processed signal at step 312, employing a Convolutional Neural Network (CNN) module 106 to measure and predict blood glucose and blood pressure levels using the extracted information at step 314, securely store the predicted results in a cloud-based database and then render the same through an API at step 316 and displaying the predicted blood glucose and blood pressure levels on a screen of the biosensor apparatus at step 318.

According to an embodiment of the present invention, the preprocessing steps comprise of scaling the signals from an original range to a new range of 0 to 255, implementing a continuous wavelet transform on the scaled signals using a Complex Gaussian Derivative Wavelet with 8th-order derivatives, obtaining coefficients and frequencies from the continuous wavelet transform, generating a scalogram image by mapping the obtained coefficients onto a pixel grid and saving the scalogram image.

$$[ signal = 255 = [ ( 255 - 0 ) * ( signalMax - signal ) / ( signalMax - signalMin ) ] .$$

$$scales = np . arrange ( np . min ( signal ) + 1 , np . max ( signal ) + 1 ) .$$

In the next step involving conversion, the continuous wavelet transform is implemented using ‘PyWavelet’.

$$coef , freqs = pywt . cwt ( signal , scales {,}^{′} cgau {8}^{′} , 33 )$$

Here, 33 is the sampling period for the frequencies output. The signals were captured with a frequency of 33 data points per second. cgau8=Complex Gaussian Derivative Wavelets with 8th-order derivatives.

$$ψ ( t ) = C {exp}^{- jt} {exp}^{- {t}^{2}}$$

where, C is an order-dependent normalization constant.

After obtaining the coefficients and frequencies from the continuous wavelet transform, the scalogram image is created and saved.

According to an embodiment of the present invention, the processing of the scalogram image in the biosensor apparatus comprises the steps of receiving a scalogram image using a receiver. The receiver 104a configured to resize the scalogram image array to a new shape of (255, 255, 3), then normalize the scalogram image array. Then outputting the processed scalogram image array using an output module.

According to an embodiment of the present invention, the measuring and predicting blood glucose and blood pressure levels by the Convolutional Neural Network (CNN) module 106 comprises the steps of receiving the preprocessed scalogram image array with an input shape of (255, 255, 3), representing width, height, and number of color channels, through an input layer. Next, applying a set of 16 filters of size 3×3 to the input image array using a convolutional layer, with a stride of 1 and valid padding, to produce an output of size (253, 253, 16). Next, applying a rectified linear unit (ReLU) activation function to the output of the convolutional layer using an activation layer, resulting in an output of size (253, 253, 16). Further performing max pooling with a window size of 2×2, a stride of 2, and valid padding on the output of the activation layer using a first max pooling layer, generating an output of size (126, 126, 16). These combinations of three layers; convolution, activation and max pooling respectively comprises a logical block, which gets repeated in subsequent operation with respective parameters.

The process further comprises repeating the logical block, mentioned in previous paragraph with the following parameters comprising utilizing a second convolutional layer 126 with 32 filters of size 5×5, a stride of 2, and valid padding, then applying a ReLU activation function to the output of the second convolutional layer 126 using a second activation layer 128. Next, performing max pooling a second max pooling layer 130 with a window size of 2×2, a stride of 2, and valid padding on the output of the second activation layer 128 using a second max pooling layer. Next, utilizing a third convolutional layer 132 with 50 filters of size 5×5, a stride of 2, and valid padding. Further, applying a ReLU activation function to the output of the third convolutional layer 132 using a third activation layer 134. And performing max pooling with a window size of 2×2, a stride of 2, and valid padding on the output of the third activation layer 134 using a third max pooling layer 136.

The method further comprises applying a first fully connected layer 140 with 1024 neurons and a ReLU activation function, incorporating a dropout layer with a dropout rate of 20% and employing a second fully connected layer 144 with 512 neurons and a ReLU activation function. Further employing a third fully connected layer 148 with 128 neurons and ReLU activation function, finally employing an output layer 150 with 3 neurons and linear activation function representing blood glucose, systolic blood pressure, and diastolic blood pressure predictions.

An apparatus for non-invasive measurement and prediction of blood glucose and blood pressure levels is disclosed.

FIG. 2 illustrates a block diagram of an embodiment of an apparatus for non-invasive measurement and prediction of blood glucose and blood pressure levels according to an embodiment of the present disclosure. The apparatus comprises of a biosensor module 102 incorporating optical spectroscopic techniques for capturing physiological signals, a microcontroller module 202 with networking capabilities for signal processing, analysis and transmission, a display screen module 108 for presenting measurement results, a LiPo battery module 204 for power supply and an enclosure module for housing and protecting the components.

According to an embodiment of the present invention, the biosensor module 102 is configured to transmit the captured physiological signals, pre-processed data, and prediction results to a cloud-based server for remote patient monitoring. This allows healthcare providers to remotely access and monitor patients' health data as healthcare IoT service.

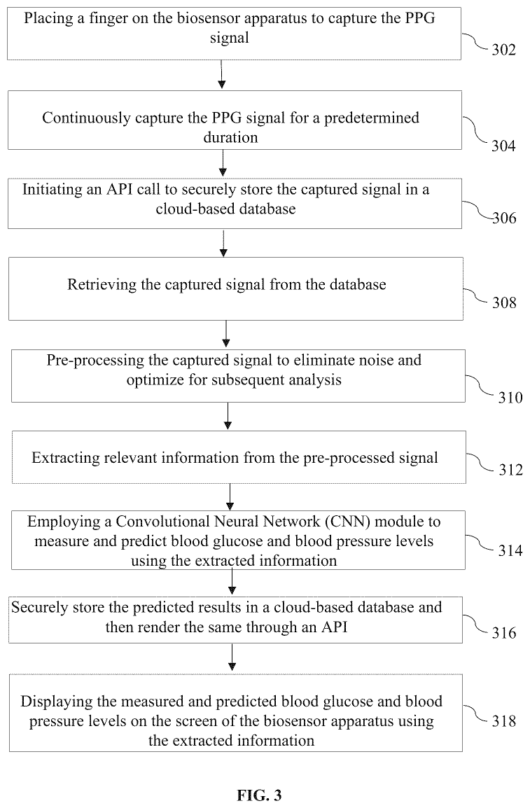

FIG. 4 illustrates a schematic diagram illustrating an emitter unit 102a and a detector unit 102b of a biosensor module 102 according to an embodiment of the present disclosure. According to an embodiment of the present invention, the biosensor module 102 further comprises internal LEDs, photodetectors, optical elements, and low-noise electronics with ambient light rejection.

According to an embodiment of the present invention, the microcontroller module 202 comprises a 32-bit, 80 MHz to 160 MHz, RTOS, integrated TCP/IP protocol, WiFi 2.4 GHZ, WPA/WPA2 security mode, default serial baud rate 115200, operating voltage: 3.3V, Maximum Working current: 240 mA with SPI, and UART communication protocols.

According to an embodiment of the present invention, the display screen module 108 comprises a OLED Display, I2C, 0.96 Inches, 128×64 resolution operating voltage 3.3V to 5.0V.

According to an embodiment of the present invention, the LiPo battery module 204 comprises a 3.7V Lithium Polymer battery with a capacity of 300 mAH.

Further, the biosensor module 102 is configured to provide healthcare IoT service in real-time monitoring of blood glucose and blood pressure levels. This is achieved by enabling continuous data capture and updating the cloud-based platform in real-time.

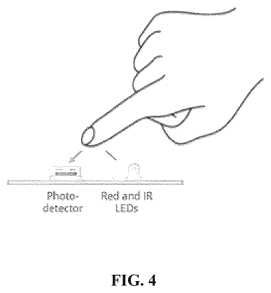

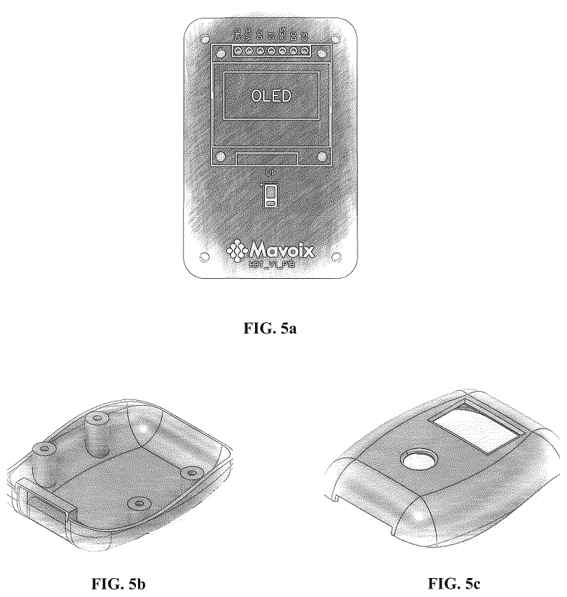

FIGS. 5a to 5c illustrates a schematic diagram illustrating an enclosure of the apparatus for non-invasive measurement and prediction of blood glucose and blood pressure levels according to an embodiment of the present disclosure. According to an embodiment of the present invention, the enclosure module is made of ABS material and comprises additional features such as buttons, switches, and ports for enhanced user interaction.

According to an embodiment of the present invention, the biosensor module 102 operates within an operating temperature range of −30° C. to 70° C.

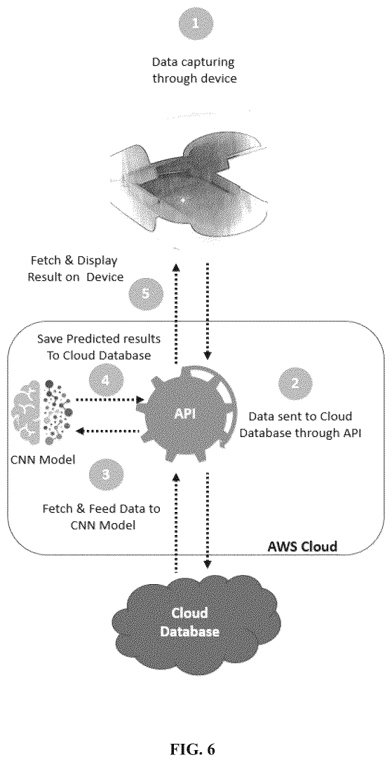

FIG. 6 illustrates a schematic diagram illustrating an overall workflow of the biosensor system 100 for non-invasive measurement and prediction of blood glucose and blood pressure levels according to an embodiment of the present disclosure. The first operation of the biosensor system 100 involves capturing physiological signals from subject. This process occurs when a finger is placed on the device sensor while it is powered on. Unlike invasive blood glucose measuring devices, this device offers a non-intrusive solution, eliminating the need for a blood sample and ensuring a comfortable experience for the user. When the device is on, upon placing the finger on the sensor, the device continuously captures the physiological signals for a brief duration, typically around 10-15 seconds. Then it initiates an API call to facilitate the safe and secure storage of the signals in a cloud-based database for further analysis.

The next operation involves retrieving the captured signal from the cloud-based database. To analyze the signal accurately, another API call is made to retrieve the signal data. Prior to analysis, the captured signal undergoes pre-processing to eliminate any potential noise and for some other necessary operations and prepare it for comprehensive analysis. This pre-processing stage ensures that the signal is clean and optimized for subsequent analysis. It is also ensured to maintain the original information intact while enhancing the quality.

Once the signal has been pre-processed, the processes within the biosensor system 100 efficiently extracts all the relevant information embedded within the signal. This step is crucial to ensure that no data loss occurs during the information extraction process. The system accurately measures and predicts vital signs using the extracted information. The AI model analyzes the signal data to derive valuable insights regarding the user's blood glucose and blood pressure level. After the vital signs have been predicted, the results are promptly displayed on the screen of the device. Prior to the display, a post-processing step may be employed to further refine the results and ensure their accuracy and clarity. This allows users to conveniently access their vital sign information in real-time, enabling them to monitor their health conditions effectively.

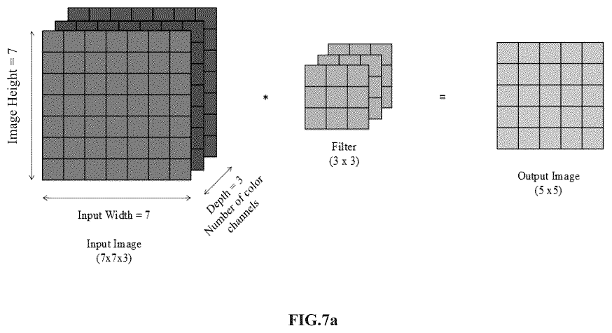

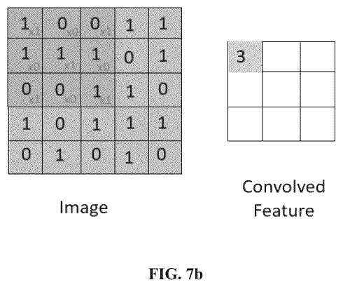

FIGS. 7a and 7b illustrates a schematic diagram illustrating the first convolution operation of a Convolutional Neural Network (CNN) module 106 according to an embodiment of the present disclosure. According to an embodiment of the present invention, the preprocessed scalogram image array is the input to the neural network and the outputs are the predicted vitals. The input shape of the image is (255, 255, 3) (width, height, numbers of color channels).

In the first convolution layer of the Convolutional Neural Network (CNN) module 106, the convolution operation on the input image is performed to extract the features from each pixel of the image.
Formula for the convolution operation is:

$$( w , h , c ) * ( f , f , c ) = [ ( \frac{w + 2 p - f}{s} + 1 ) \cdot ( \frac{h + 2 p - f}{s} + 1 ) , {c}^{′} ]$$

Where,
   w=width of the input image
   h=height of the input image
   c=the number of color channels of the input image
   f=width & height of the convolution filter
   c′=the member of convolution filters
   p=padding, 0 for ‘valid’ and 1 for ‘same’
   s=stride (steps to move kernel/filter)

As further illustrated in FIG. 7b shows a sample input image and the convolved feature, in the first convolution layer of the Convolutional Neural Network (CNN) module 106, 16 filters of size 3×3, the stride of 1, and ‘valid’ padding is applied. The obtained output width and height is: ((255+(2*0)−3)/1)+1=253. The output of the first convolution layer is (253, 253, 16).

According to an embodiment of the present invention, in the activation layers, the ReLU activation function—which is a non-linear function—is applied to the output of every convolution layer and dense layers except the output layer. The reason to use a non-linear activation function is to avoid the neural network being a regression model. If we apply a linear activation function to the hidden layers, all the layers will behave the same way because the composition of two linear function is linear function itself.

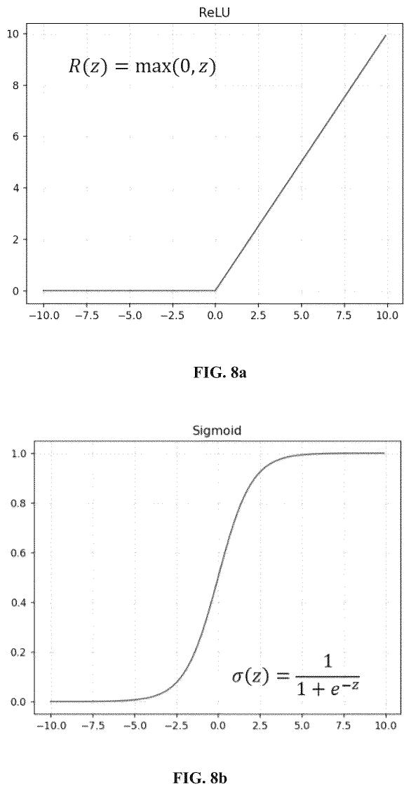

FIGS. 8a and 8b illustrates a graphical representation of output of the ReLU and Sigmoid activation functions. ReLU activation function is less computationally expensive than ‘tanh’ and ‘sigmoid’ because it involves simpler mathematical operations. Therefore, ReLU activation function is used.
Formula (ReLU activation):

$$A ( x ) = max ( 0 , x )$$

It gives an output x if x is positive and 0 otherwise. Further, the Output of the first activation layer 122 is (253, 253, 16).

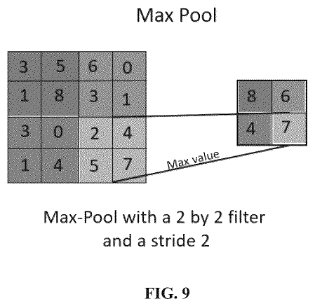

FIG. 9 illustrates a schematic diagram illustrating a max pooling operation of a Pooling layer of Convolutional Neural Network (CNN) module 106 according to an embodiment of the present disclosure. According to an embodiment of the present invention, it employs window size of 2×2, a stride of 2, and ‘valid’ padding. The formula for computing the output is the same as in the convolution layer. The output of the first max pooling layer 124 is (126, 126, 16).

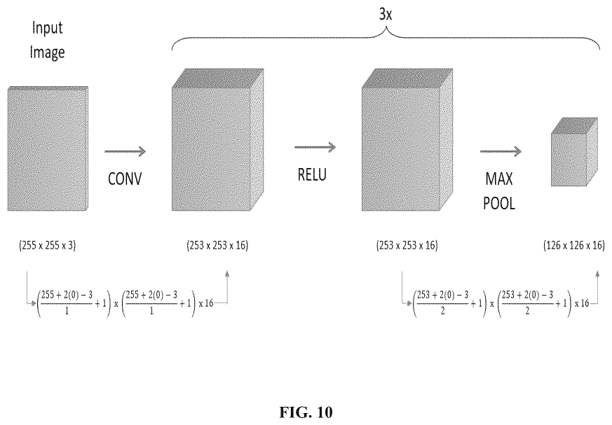

FIG. 10 illustrates a schematic diagram illustrating blocks of a convolution layer, a ReLU activation layer and a max pooling layer according to an embodiment of the present disclosure. According to an embodiment of the present invention, the remaining layers are represented as a block. Next, add two sets of these three layers as follows:
Block 1
Fourth Layer: Second Convolution Layer
32 filters of size 5×5, a stride of 2, and padding is ‘valid’
Fifth Layer: Second ReLU Activation Layer
Sixth Layer: Second MaxPooling Layer
window size of 2×2, a stride of 2, padding is ‘valid’
Block 2
Seventh Layer: Third Convolution Layer
50 filters of size 5×5, a stride of 2, and padding is ‘valid’
Eighth Layer: Third ReLU Activation Layer
Ninth Layer: Third MaxPooling Layer
window size of 2×2, a stride of 2, padding is ‘valid’

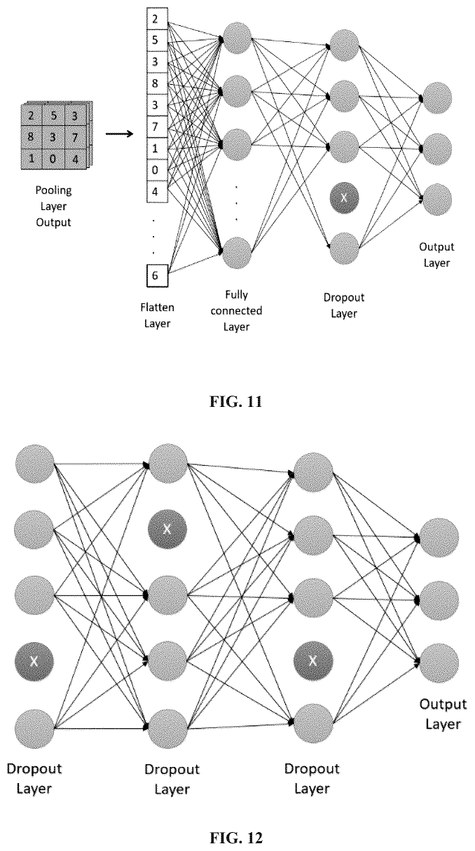

FIG. 11 illustrates a schematic diagram illustrating the connection of a flatten layer 138, fully connected layer, dropout layer and output layer. According to an embodiment of the present invention, if a fully connected layer is introduced, the model is provided with the ability to mix information, since every single neuron has a connection to every single one in the next layer, now there is a flow of information between each input dimension (pixel location) and each output class, thus the decision is based truly on the whole image.
Tenth Layer: Flatten Layer
Eleventh Layer: First Fully Connected (Dense) Layer
1024 neurons, ‘normal’ kernel initializer, ‘relu’ activation

FIG. 12 illustrates a schematic diagram illustrating the connection of dropout layers according to an embodiment of the present disclosure. According to an embodiment of the present invention, there are two dropout layers with 20% probability (to drop 20% of the neuron values) has been added after the first and the second Dense layer respectively. Dropout layers helps prevent overfitting. It acts as a mask, eliminating some neurons' contributions to the subsequent layer while maintaining the functionality of all other neurons.
Twelfth Layer: First Dropout Layer
with 20% probability (to drop 20% of the neuron values)
Thirteenth Layer: Second Fully Connected (Dense) Layer
512 neurons, ‘normal’ kernel initializer, ‘relu’ activation
Fourteenth Layer: Second Dropout Layer
with 20% probability (to drop 20% of the neuron values)
Fifteenth Layer: Third Fully Connected (Dense) Layer
128 neurons, ‘normal’ kernel initializer, ‘relu’ activation
Sixteenth Layer: Output Layer
3 neurons, ‘linear’ activation

Finally, the output layer 150 has three neurons, exactly the same number of vitals to predict (blood glucose, systolic blood pressure, diastolic blood pressure).
Output shape of the final layer: (3,)
Output=[[blood glucose, systolic blood pressure, diastolic blood pressure]]

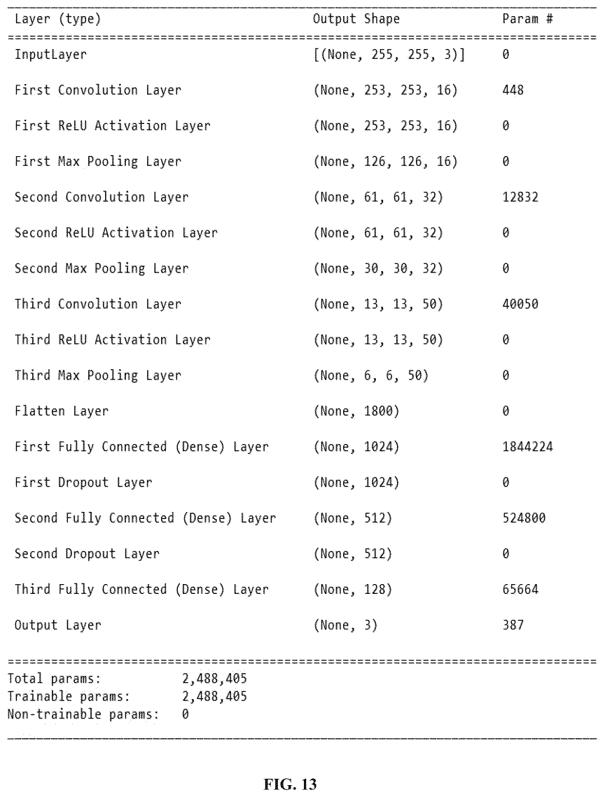

FIG. 13 is a table representing all the layers, their return shapes and the number of training parameters according to an embodiment of the present disclosure. According to an embodiment of the present invention, the hyperparameters tuning and model training comprises of:

### Optimizer:

opt=tf.keras.optimizers.Nadam(learning_rate=0.001,beta_1=0.9,beta_2=0.999,epsilon=1e−07,decay=None,clipnorm=None,clipvalue=None,global_clipnorm=None,name=“Nadam”)

### Loss Function:

loss=tf.keras.losses.MeanSquaredError(reduction=“auto”,name=“mean_squared_error”)

### Others:

Metrics: Mean Square Error
Epochs: 1600
Batch size: 64

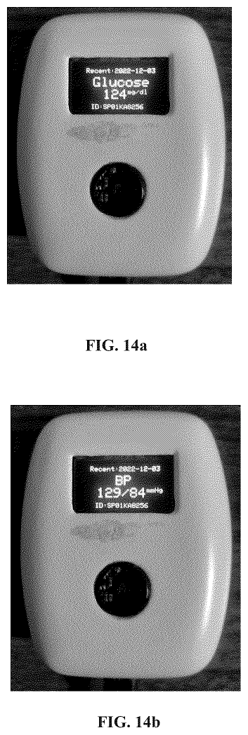

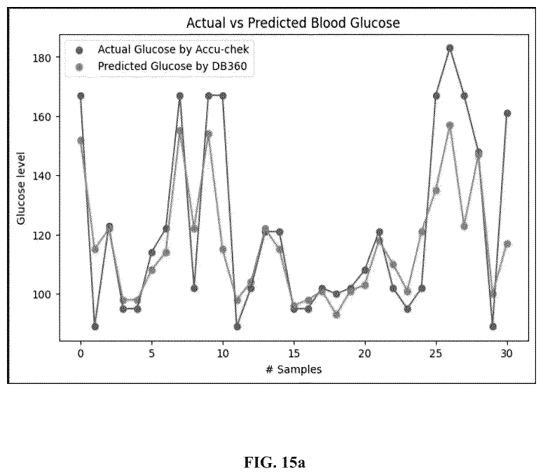

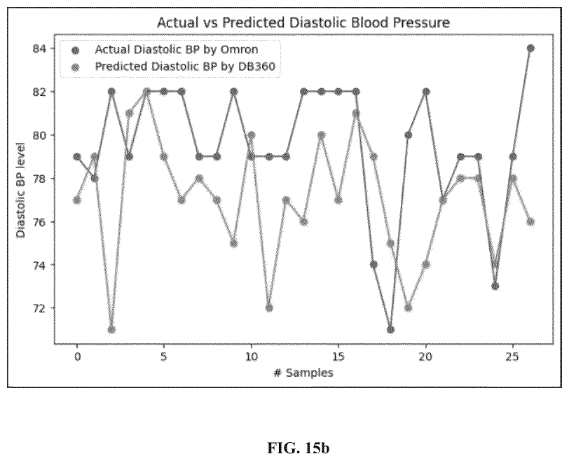

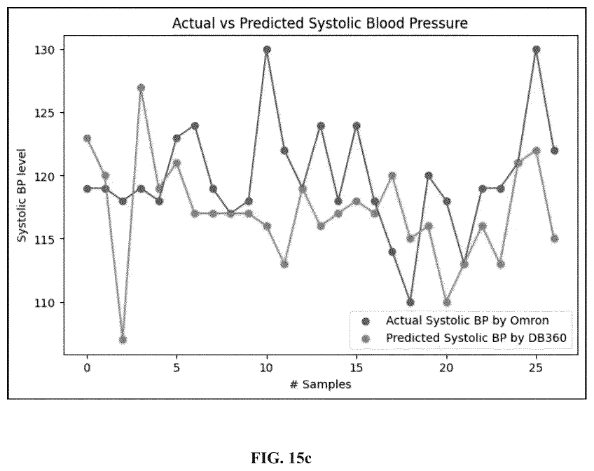

FIGS. 14a and 14b illustrates a protype of the apparatus for non-invasive measurement and prediction of blood glucose and blood pressure levels according to an embodiment of the present disclosure. FIGS. 15a to 15c is a graphical representation of comparison of levels of blood glucose and blood pressure between the existing products in the market and the apparatus of the present invention. During the live testing, subjects interacted with the device in a suitable environment. Data from each subject was collected and processed using the biosensor system 100. The system's predictions or classifications were obtained, and performance metrics such as, precision, recall, and F1-score were calculated to assess the model's effectiveness. The report emphasizes the importance of analyzing challenges or limitations encountered during the live testing, such as variations in data quality, subject-specific characteristics, and real-world conditions.

The validation process involved rigorous testing of the apparatus's ability to measure and predict blood glucose and blood pressure levels. A standardized protocol was followed, and actual measurements were taken using established devices (Accu-check [trademark] active for blood glucose and Omron [trademark] for blood pressure). A total of 31 test samples were collected over a period of 3 months. The validation results indicate that the device accurately predicted blood glucose and blood pressure within their respective measuring ranges. The FIG.15a to 15c presents the graphs displaying the test results for each sample of every vital.

Based on the validation results, it can be concluded that the apparatus demonstrates high accuracy in measuring and predicting blood glucose and blood pressure. The sample standard deviation, representing the difference between the actual and predicted values, was found to be less than 15 percent for a significant majority of the samples. This indicates a high level of accuracy in the device's measurements.

Overall, the biosensor apparatus allows for non-invasive measurement and prediction of blood glucose and blood pressure levels. It eliminates the need for traditional invasive methods such as finger pricking, making it more comfortable and convenient for users. The apparatus incorporates a biosensor unit that captures physiological signals. It preprocesses the captured signals to eliminate noise and optimize them for subsequent analysis. The biosensor module 102 incorporates optical spectroscopic techniques for capturing physiological signals. It uses multiple light sources emitting light of different wavelengths and photodetectors sensitive to the emitted light wavelengths, allowing for accurate signal detection. The apparatus utilizes a processing technique, which involves extracting relevant information from the pre-processed physiological signals. This technique includes frequency and time-domain analysis to identify features relevant to blood glucose and blood pressure levels.

Furthermore, the apparatus employs a CNN module 106 for measuring and predicting blood glucose and blood pressure levels based on the extracted information. The CNN module 106 allows for efficient and accurate analysis of the signal data. The apparatus includes a display unit with a screen and a user interface module 110 for receiving user commands and displaying information. This enables users to interact with the device and view the predicted vital signs. The apparatus utilizes cloud-based computing capabilities for secure storage of captured signals and seamless data processing and analysis. This ensures timely and accurate results and enables data sharing and remote patient monitoring capabilities.

Further, the validation results demonstrate the effectiveness and reliability of the biosensor apparatus in measuring and predicting blood glucose and blood pressure levels. The device shows a high level of accuracy, with a low deviation between the actual and predicted values. The biosensor apparatus provides a non-invasive and user-friendly solution for monitoring blood glucose and blood pressure. It eliminates the pain and discomfort associated with traditional methods, improving the overall user experience. Also, the device follows a modular design approach which allows for scalability, customization, and easy maintenance of the device.

The present description has been shown and described with reference to the foregoing examples. It is understood, however, that other forms, details, and examples can be made without departing from the spirit and scope of the present subject matter.

We claim:

1. A biosensor system for non-invasive measurement and prediction of blood glucose and blood pressure levels, comprising:
a biosensor module configured to capture signals comprising physiological signals, and preprocess the captured signals to eliminate noise and optimize for subsequent analysis;
a processing module configured to extract relevant information from the preprocessed physiological signals using a processing technique;
a convolutional neural network (CNN) module configured to measure and predict blood glucose and blood pressure levels based on the extracted information;
a display module comprising a screen;
a user interface module configured to receive one or more user commands and display information on the screen; and
a control module configured to control the operation of the biosensor apparatus.

2. The biosensor system as claimed in claim 1, wherein the biosensor module incorporates optical spectroscopic techniques for capturing physiological signals, comprises:
an emitter unit configured to emit light wavelengths in the range of 660 nm to 1100 nm onto a target region of a user's body;
a detector unit positioned adjacent to the emitter unit and configured to receive the light after it has passed through the target region;
a signal processing unit operatively coupled to the detector unit, configured to process the received light signals and generate a physiological signal;
a pre-processing module configured to eliminate noise and optimize the physiological signal for subsequent analysis; and
wherein the emitter unit comprises of a plurality light sources and the detector unit comprises one or more photodetectors sensitive to the emitted light wavelengths;
wherein the signal processing unit further comprises a spectral analysis module for analyzing the received light signals to extract physiological information.

3. The biosensor system as claimed in claim 1, wherein the biosensor module preprocesses the signals to generate scaled signals for generating a scalogram image, the preprocessing steps comprises of:
scaling the signals from an original range to a new range of 0 to 255;
implementing a continuous wavelet transform on the scaled signals using a Complex Gaussian Derivative Wavelet with 8th-order derivatives;
obtaining coefficients and frequencies from the continuous wavelet transform;
generating a scalogram image by mapping the obtained coefficients onto a pixel grid; and
saving the scalogram image.

4. The biosensor system as claimed in claim 1, wherein the processing technique implemented by the processing module is configured to process the scalogram image, comprising:
a receiver configured to receive the scalogram image;
a resizer configured to resize the scalogram image array to the shape of (255, 255, 3);
a normalizer configured to normalize the resized scalogram image array;
an output module configured to output the processed scalogram image array.

5. The biosensor system as claimed in claim 1, wherein the Convolutional Neural Network (CNN) module is configured to measure and predict blood glucose and blood pressure levels, comprises:
an input layer configured to receive a preprocessed scalogram image array having an input shape of (255, 255, 3), representing width, height, and number of color channels;
a first convolutional layer configured to apply a set of 16 filters of size 3×3 to the input image array, with a stride of 1 and valid padding, producing an output of size (253, 253, 16);
a first activation layer configured to apply a rectified linear unit (ReLU) activation function to the output of the first convolutional layer, producing an output of size (253, 253, 16);
a first max pooling layer configured to perform max pooling with a window size of 2×2, a stride of 2, and valid padding on the output of the first activation layer, producing an output of size (126, 126, 16);
a second convolutional layer configured to apply a set of 32 filters of size 5×5 to the output of the first max pooling layer, with a stride of 2 and valid padding, producing an output of size (61, 61, 32);
a second activation layer configured to apply a rectified linear unit (ReLU) activation function to the output of the second convolutional layer, producing an output of size (61, 61, 32);
a second max pooling layer configured to perform max pooling with a window size of 2×2, a stride of 2, and valid padding on the output of the second activation layer, producing an output of size (30, 30, 32);
a third convolutional layer configured to apply a set of 50 filters of size 5×5 to the output of the second max pooling layer, with a stride of 2 and valid padding, producing an output of size (13, 13, 50);
a third activation layer configured to apply a rectified linear unit (ReLU) activation function to the output of the third convolutional layer, producing an output of size (13, 13, 50);
a third max pooling layer configured to perform max pooling with a window size of 2×2, a stride of 2, and valid padding on the output of the third activation layer, producing an output of size (6, 6, 50);
a flatten layer configured to flatten the output of the third max pooling layer producing a output of shape (1800,);
a first fully connected layer with 1024 neurons and ReLU activation function;
a first dropout layer with a dropout rate of 20%;
a second fully connected layer with 512 neurons and ReLU activation function;
a second dropout layer with a dropout rate of 20%;
a third fully connected layer with 128 neurons and ReLU activation function;
an output layer with three neurons and linear activation function, representing blood glucose, systolic blood pressure, and diastolic blood pressure predictions.

6. A method for non-invasive measurement and prediction of blood glucose and blood pressure levels using a physiological signals captured by a biosensor apparatus, comprising the steps of:
a) placing a finger on the biosensor apparatus to capture the physiological signals;
b) continuously capturing the physiological signals for a predetermined duration;
c) initiating an API call to securely store the captured signal in a cloud-based database;
d) retrieving the captured signal from the cloud-based database;
e) pre-processing the captured signal to eliminate noise and optimize for subsequent analysis;
f) extracting relevant information from the pre-processed signal;
g) employing a Convolutional Neural Network (CNN) module to measure and predict blood glucose and blood pressure levels using the extracted information; and
h) displaying the predicted blood glucose and blood pressure levels on a screen of the biosensor apparatus.

7. The method as claimed in claim 6, wherein the preprocessing steps comprise of:
scaling the signals from an original range to a new range of 0 to 255;
implementing a continuous wavelet transform on the scaled signals using a Complex Gaussian Derivative Wavelet with 8th-order derivatives;
obtaining coefficients and frequencies from the continuous wavelet transform;
generating a scalogram image by mapping the obtained coefficients onto a pixel grid; and
saving the scalogram image.

8. The method as claimed in claim 6, wherein the processing technique by the processing module to process the scalogram image, comprises:
receiving a scalogram image array using a receiver;
resizing the scaled scalogram image array to the shape of (255, 255, 3);
normalizing the resized scalogram image array using a normalizer; and
outputting the processed scalogram image array using an output module.

9. The method as claimed in claim 6, wherein measuring and predicting blood glucose and blood pressure levels by the Convolutional Neural Network (CNN) module comprises the steps of:
a) receiving the preprocessed scalogram image array with an input shape of (255, 255, 3), representing width, height, and number of color channels, through an input layer;
b) applying a set of 16 filters of size 3×3 to the input image array using a first convolutional layer, with a stride of 1 and valid padding, to produce an output of size (253, 253, 16);
c) applying a rectified linear unit (ReLU) activation function to the output of the convolutional layer using an activation layer, resulting in an output of size (253, 253, 16);
d) performing max pooling with a window size of 2×2, a stride of 2, and valid padding on the output of the activation layer using a first max pooling layer, generating an output of size (126, 126, 16);
e) repeating steps b) to d) with the following parameters:
i. utilizing a second convolutional layer with 32 filters of size 5×5, a stride of 2, and valid padding;
ii. applying a ReLU activation function to the output of the second convolutional layer using a second activation layer;
iii. performing max pooling with a window size of 2×2, a stride of 2, and valid padding on the output of the second activation layer using a second max pooling layer;
iv. utilizing a third convolutional layer with 50 filters of size 5×5, a stride of 2, and valid padding;
v. applying a ReLU activation function to the output of the third convolutional layer using a third activation layer;
vi. performing max pooling with a window size of 2×2, a stride of 2, and valid padding on the output of the third activation layer using a third max pooling layer;
f) incorporating a flatten layer to flatten the output of the third max pooling layer;
g) applying a first fully connected layer with 1024 neurons and a ReLU activation function;
h) incorporating a first dropout layer with a dropout rate of 20%;
i) employing a second fully connected layer with 512 neurons and a ReLU activation function; and
j) incorporating second dropout layer with a dropout rate of 20%;
k) employing a third fully connected layer with 128 neurons and a ReLU activation function; and
l) employing an output layer with 3 neurons and linear activation function, representing blood glucose, systolic blood pressure, and diastolic blood pressure predictions.

10. An apparatus for non-invasive measurement and prediction of blood glucose and blood pressure levels, comprising:
a biosensor module incorporating optical spectroscopic techniques for capturing physiological signals;
a microcontroller module with networking capability for signal processing, analysis and transmission;
a display screen module for presenting measurement results;
a LiPo battery module for power supply; and
an enclosure for housing and protecting the components.

11. The apparatus as claimed in claim 10, wherein the biosensor module further comprises internal LEDs, photodetectors, optical elements, and low-noise electronics with ambient light rejection.

12. The apparatus as claimed in claim 10, wherein the microcontroller module comprises a 32-bit, 80 MHz to 160 MHz, RTOS, integrated TCP/IP protocol, WiFi 2.4 GHz, WPA/WPA2 security mode, default serial baud rate 115200, operating voltage: 3.3V, Maximum Working current: 240 mA with SPI, and UART communication protocols.

13. The apparatus as claimed in claim 10, wherein the display screen module comprises a OLED Display, I2C, 0.96 Inches, 128×64 resolution operating voltage 3.3V to 5.0V.

14. The apparatus as claimed in claim 10, wherein the LiPo battery module comprises a 3.7V Lithium Polymer battery with a capacity of 300 mAH.

15. The apparatus as claimed in claim 10, wherein the enclosure is made of ABS material and comprises additional features such as buttons, switches, and ports for enhanced user interaction.

16. The apparatus as claimed in claim 10, wherein the biosensor module operates within an operating temperature range of −30° C. to 70° C.

17. The apparatus as claimed in claim 10, wherein the biosensor module r unis configured to transmit the captured physiological signals, pre-processed data, and prediction results to a cloud-based server for remote patient monitoring, IoMT (Internet of Medical Things), wearables, smartphones, and fitness assistance devices.
